(19) United States
(12) Patent Application Publication
(10) Pub. No.: US 2025/0090592 A1
(43) Pub. Date: Mar. 20, 2025

(54) Treatment of Mitochondrial Disease with Autologous Expanded Mesenchymal Stem Cell Therapy

(71) Applicants: Elliot B. Landers, MD., Rancho Mirage, CA (US); Christopher James, Portland, OR (US)

(72) Inventors: Elliot B. Landers, MD., Rancho Mirage, CA (US); Christopher James, Portland, OR (US)

(21) Appl. No.: 18/890,553

(22) Filed: Sep. 19, 2024

Related U.S. Application Data
(60) Provisional application No. 63/539,297, filed on Sep. 19, 2023.

(51) Int. Cl. A61K 35/28; C12N 5/0775

(52) U.S. Cl. CPC A61K 35/28; C12N 5/0667

(57) ABSTRACT

A method for providing healthy mitochondria for mitochondrial donation MSCs includes (1) harvesting the patient's mesenchymal stem cells (HSMCs) from that patient's adipose tissue by a liposuction procedure, (2) separating the MSCs having a specified Cluster of Differentiation (CD Markers) from the adipose environment; (3) cryofreezing the MSCs to a state where metabolic activity has ceased; (4) recovering by a gradual warming protocol of MSCs to viability; (4) expansion of viable MSCs in a special growth medium; (5) preparing the expanded MSCs for administration by several known methods e.g., size filtration; (6) intravenously administrating the MSCs; (7) administrating the MSCs by intramuscular injection; (8) and repeating intravenous and intramuscular administration of the MSCs every 60 to 90 days.

### PRIORITY CLAIM

This application claims priority to provisional application 63/359,297 filed on Sep. 19, 2023 and is incorporated by reference for all purposes

### BACKGROUND

This invention relates to the use of mesenchymal stem cells (MSCs) for the treatment of disease. Mesenchymal stem cells have been used in clinical applications for mitigating tissue injury and facilitating tissue healing and regeneration. The action of MSCs that provide therapeutic value was not settled, but the donation of mitochondria by tubular transfer, cell fission, or fusion or through exosome transfers of MSC has been proposed.

For example, an early clinical experiment was mitochondrial transplantation to treat myocardialischemia—reperfusion injury in pediatric patients. Mitochondria were isolated from the patients' non-ischemic skeletal muscles and injected directly into the injured myocardium. The treatment improved ventricular function, and there were no adverse complications such as arrhythmia, intramyocardial hematoma, or scarring.

The present invention is a therapy that uses autologous adipose-derived laboratory-expanded mesenchymal stem cells to improve the prevalence, health, and function of mitochondria in Duchenne Muscular Dystrophy (DMD) patients. Heretofore, the use of donor cells with intact dystrophin has been regarded as necessary for therapy under a theory of the disease that replacement of dystrophin (the genetically damaged protein) in the cells of a Duchenne patient is required for therapeutic value. What is innovative is that all other regerative medicine treatments were aimed at restoring or improving the muscle cells and we are targeting the bio-energetics of the target cells to restore and re-populate mitochondria, making this a “sub-cellular” therapy and not just a cellular therapy.

The former proposition does not consider that low-prevalence and/or low-functioning mitochondria might be a significant aspect of the disease. There is research that shows a pronounced mitochondrial deficit in DMD patients. Other research shows that stem cell functions, particularly stem cell replication and division, require the greatest level of cellular energy in human cells. Thus, deficient cellular energy may be an aspect of pathology in Duchenne Muscular Dystrophy and improving mitochondrial prevalence and/or function may provide a useful therapy for the disease. What is innovative is that all other regenerative medicine treatments were aimed at restoring or improving the muscle cells and the present invention targets the bio-energetics of the target cells to restore and re-populate mitochondria, making this a “sub-cellular” therapy and not just a cellular therapy.

Moreover, the replacement of non-functional, low-functioning, or dysfunctional mitochondria by transplantation of healthy mitochondria into injured cells is believed to potentially be a universal solution for the treatment of mitochondrial deficiency of different etiologies. Delivery of even a few healthy mitochondria from autologous or allogenic donor mesenchymal stem cells can lead to the sustained restoration of quantitative and qualitative mitochondrial function in a recipient cell. Thus, there exists a need for a therapy that provides healthy mitochondria for the treatment of DMD and any disease where mitochondrial dysfunction is present. The properties resident in the MSC utilized in the invention include, the ability to deliver both the IMF and SSS mitochondria, the ability to pass through nanotubes, the ability to migrate along arterial vessels the ability to concentrate in lung tissues, the ability to cross the blood-brain barrier (BBB), reconstitution of mitochondria in neural cells the ability to reconstitute mitochondria in nerve cells (glial, Schwann, and others) and the ability to signal other Fibroblast Growth Factor families.

The pathophysiology of Duchenne Muscular Dystrophy is similar to that in other well-characterized diseases, including all other forms of Muscular Dystrophy (Becker's Limb-Girdle, etc.) cardiovascular disease, Alzheimer's, chronic obstructive pulmonary disease, dementia, aging, ischemic heart disease. Because FGF 21 has a short half-life and is excreted by skeletal muscle, its elevation from the invention protocol indicates systemic influence that would ameliorate and be therapeutic in the disease above and any other metabolic disease.

### BRIEF DESCRIPTION OF THE DRAWINGS

FIG. 1 lists graphs of fibroblast growth factor 21 concentration versus time.

FIGS. 2A to 2C lists a longitudinal study of fibroblast growth factor 21 over 5 years.

### DETAILED DESCRIPTION

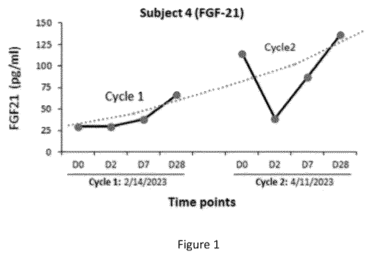

The invention provides a source of healthy mitochondria and mitochondrial function using autologous MSCs. The MSCs, once expanded and processed, are administered to the patient. The donation results are monitored by measuring the concentration of fibroblast growth factor 21 (FGF 21), which serves as a proxy measure of mitochondrial function in humans. An increase in serum FGR-21 is evidence of positive improvement in mitochondrial health and function in DMD patients. The results shown in FIG. 1 indicate a 480% increase in serum FGF-21 over an eighty-five-day course of treatment. The MSCs are autologous, i.e., sourced from the patient process using the method described below and then administered to the patient. Because the patient's own expanded MSCs are used, they are immune privileged and do not cause host vs. graft or graft vs. host disease or similar rejection that may be associated with allogenic cell donations.

In the best mode of the invention, the method for providing healthy mitochondria for mitochondrial donation from MSCs includes (1) harvesting the patient's mesenchymal stem cells (HSMCs) from that patient's adipose tissue by a liposuction procedure, (2) separating the MSCs having a specified Cluster of Differentiation (CD Markers) from the adipose environment; (3) cryofreezing the MSCs to a state where metabolic activity has ceased; (4) recovering by a gradual warming protocol of MSCs to viability; (4) expansion of viable MSCs in a special growth medium; (5) preparing the expanded MSCs for administration by several known methods e.g., size filtration; (6) intravenously administrating the MSCs; (7) administrating the MSCs by intramuscular injection; (8) and repeating intravenous and intramuscular administration of the MSCs every 60 to 90 days, (9)

Collecting a patient's adipose, separation of MSCs, cryofreezing, and cell expansion is well known in the art, and for brevity, the different methods will not be discussed here. Similarly, the administration of therapeutics intravenously and intra-muscular injection is well-known and widely practiced in the art, and brevity will not be discussed here. Nonetheless, any accepted method of the former, now known or conceived of later, is acceptable.

In the preferred embodiment, the embodiment MCS cells are expanded to 60 million cells. Then, 50 million cells are administered intravenously to a patient. The other 10 million cells are administered to the same patient by intramuscular injection. In a first embodiment administration of GCSF 12 twenty-four hours in advance, intravenous infusion and intramuscular injection. In a second embodiment, the administration of GCSF 12 and mitochondrial nutraceuticals are given before and after administration of MSCs.

A third embodiment of the invention is a method for increasing serum concentration of FGF-21 in DMD patients by: (1) harvesting the patient's mesenchymal stem cells (HSMCs) from that patient's adipose tissue by a liposuction procedure; (2) separating the MSCs having a specified Cluster of Differentiation (CD Marker) from the adipose environment; (3) cryofreezing the MSCs to a state where metabolic activity has ceased; (4) recovering by a gradual warming protocol of MSCs to viability; (4) expansion of viable HMSCs in a special growth medium to a concentration of 60 million cells; (5) preparing the expanded MSCs for administration by a number of known methods e.g., size filtration; (6) intravenously administrating 50 million of the HMSCs to the DMD patient; (7) administrating the remaining 10 million MSCs by intramuscular injections to the same patient comprising a series of 8 injections: 2 to the dorsal and ventral large muscle groups of the upper extremity, and 2 to the dorsal and ventral lower arm; (8) and repeating intravenous and intramuscular administration of the MSCs every 60 to 90 days, (9) measuring the increase in serum concentration of FGF 21 over the course of treatment.

### Example

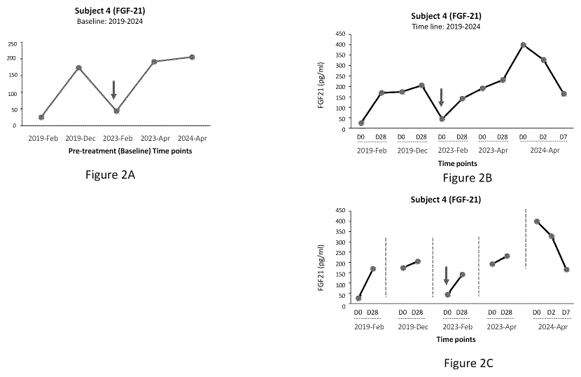

FIGS. 2A to 2C show the results of a fibroblast growth factor 21 (FGF 21) in a Longitudinal study over 5 years in subject 4. A gradual increment of baseline FGF 21 was observed when stem cells were transfused approximately in yearly intervals. Generally, the FGF21 also showed substantial increase at 28-day post ADSVF transfusion. FGF 21 level was reduced when there was longer interval of more than 2-3 years (see the reduction at 2023-Feb time point after 3 year-gap (noted by red arrows).

Applicant(s) reserves the right to submit claims directed to combinations and subcombinations of the disclosed inventions that are believed to be novel and non-obvious. Inventions embodied in other combinations and sub-combinations of features, functions, elements, and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

We claim:

1. A method for increasing the number and lifespan of a patient's mesenchymal stem cells by administration of laboratory expanded autologous mesenchymal stem cells, by:
(a) harvesting the patient's mesenchymal stem cells (HSMCs) from that patient's adipose tissue obtained by a liposuction procedure;
(b) separating the MSCs having a specified Cluster of Differentiation (CD Marker) from the adipose environment;
(c) cryofreezing the MSCs to a state where metabolic activity has ceased;
(d) recovering by a gradual warming protocol of MSCs to viability;
(e) expansion of viable HMSCs in a special growth medium to a concentration of at least 60 million cells;
(f) preparing the expanded MSCs for administration; and
(g) administrating the HMSCs to the DMD patient.

2. The method of claim 1 where Fibroblast Growth Factor (FGF) 21 is increased in the patient's serum, organs or tissue cells by reason of increased bodily generation, increased circulation, and changed properties of utilization in the patient.

3. The method of claim 1 where Fibroblast Growth Factor (FGF) 21 is increased in the patient's serum, organs or tissue cells by reason of increased bodily generation and increased circulation and changed properties of utilization by the human body.

4. The method pf claim 1 where one or more a metabolic respiratory chain (“RTCs”) functions, capacities or performance is improved in function generally when damaged or dysfunctional from disease.

5. The method of claim 1 where cells expressing CD 133 and related cells associated with Canver growth or disease are suppressed in the patient's serum, organs or tissue cells by increased volume of HMAC expanded cells.

6. The method of claim 1, where the HMSCs are intravenously administered to the DMD patient; and concurrently a separate quantity of HMSC are administered by injection.

7. The method of claim 6 where the intramuscular injections are administered in a series of 8 injections.

8. The method of claim 1 where injections are administered to a patients muscles, nerves, or organs that have a likelihood of mitochondrial or FGF 21 deficit.

9. The method of claim 1 where the HMSCs are administered intravenously and by injection repeatedly in time intervals that allow a baseline performance to improve and compound commutatively, at a minimum interval of 45 days.
